(19) United States
(12) Patent Application Publication
(10) Pub. No.: US 2016/0047748 A1
(43) Pub. Date: Feb. 18, 2016

(54) FLUORESCENT PROBE

(71) Applicant: THE UNIVERSITY OF TOKYO, Bunkyo-ku, Tokyo (JP)

(72) Inventors: Tetsuo NAGANO, Tokyo (JP); Kenjiro HANAOKA, Tokyo (JP); Kazuhisa HIRABAYASHI, Tokyo (JP)

(73) Assignee: THE UNIVERSITY OF TOKYO, Bunkyo-ku, Tokyo (JP)

(21) Appl. No.: 14/771,933

(22) PCT Filed: Mar. 4, 2014
(86) PCT No.: PCT/JP2014/055483

§ 371 Date: Oct. 21, 2015

(30) Foreign Application Priority Data
Mar. 4, 2013 (JP) 2013-042346

(51) Int. Cl. G01N 21/64; G01N 33/20; C07F 7/10

(52) U.S. Cl. CPC G01N 21/6428; C07F 7/10; G01N 33/20; G01N 21/645

(57) ABSTRACT

[Problem] To provide a fluorescent probe having a novel fluorophore. [Solution] A compound represented by general formula (I), or a salt thereof.

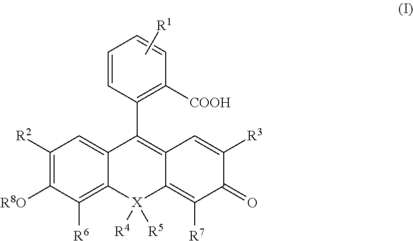

### TECHNICAL FIELD

The present invention relates to a fluorescent probe having a novel fluorophore.

### BACKGROUND ART

Fluorescein is a molecule reported in 1871 and has been widely used as a pH indicator and a labeling dye because it has high water-solubility and high fluorescence quantum yield. After calcium probes having fluorescein as a matrix were developed, there have been provided many highly sensitive fluorescence-activation probes that use intramolecular photoinduced electron transfer (PeT) and decyclization and cyclization of spiro rings or the like. In particular, probes that use intramolecular PeT are designed with consideration given to the oxidation potential of a benzene ring in fluorescein, whereby fluorescence activation can be achieved before and after a substance to be measured has been captured and the substance can be measured with high sensitivity.

Conventional fluorescent probes having a rhodamine matrix as a fluorescent dye and being capable of red-colored bio-imaging are known, and Rhod-2 and other calcium probes are being used as probes in which intramolecular PeT is utilized. However, rhodamine has an amino group in the molecule and therefore has a drawback in that it is cationic in vivo and readily accumulates in specific organelle, in particular in mitochondria.

On the other hand, there are essentially no reports related to the structural modification of the oxygen atom at position 10 of a xanthene ring of fluorescein, and the optical characteristics of a compound resulting from substituting with another atom the oxygen atom at position 10 are unknown. There are already reported a compound (TMDHS) resulting from substituting with a silicon atom the oxygen atom of pyronin Y (PY), which is the base backbone of rhodamine, and application of the compound to a fluorescent probe (Best, Q., et al., Pacifichem 2010, Presentation No. 2335, 19 Dec. 2010; Koide, Yuichiro, et al., 4th Meeting of the Japanese Society for Molecular Imaging, Presentation No. P8-9, 14 May 2009), but the fluorescence characteristics remain unknown in compounds resulting from substituting with a silicon atom the oxygen atom at position 10 of a xanthene ring of fluorescein.

The present inventors found that introducing a group capable of capturing a substance to be measured (may hereinafter be referred to as a “capturing group” in the present specification) on a benzene ring of a compound resulting from substituting with a silicon atom the oxygen atom at position 10 of a xanthene ring in a fluorescein backbone will thereby induce intramolecular PeT before and after capture of the substance to be measured and make it possible to turn fluorescence off and on (see Patent Reference 1). In particular, the present inventors showed that a compound (e.g., CaTM-2-AM, or the like) obtained by introducing a capturing group for capturing calcium ions in a benzene ring positioned in position 9 of a xanthene ring is effective as a calcium ion probe.

However, CaTM-2-AM disclosed in Patent Reference 1 has poor solubility and does not dissolve in a buffer; therefore, a surfactant must be added during cell imaging. Since aggregates are formed when a surfactant is not added, it was confirmed that the sensitivity of cell imaging is poor and insufficient for obtaining highly sensitive cell imaging.

### PRIOR ART REFERENCES

### Patent References

Patent Reference 1: WO 2012/111817

### Non-Patent References

Non-Patent Reference 1: Best, Q., et al., Pacifichem 2010, Presentation No. 2335, 19 Dec. 2010
Non-Patent Reference 2: Koide, Yuichiro, et al., 4th Meeting of the Japanese Society for Molecular Imaging, Presentation No. P8-9, 14 May 2009

### SUMMARY OF THE INVENTION

### Problems to be Solved by the Invention

An object of the present invention is to provide a fluorescent probe having a novel fluorophore.

More specifically, an object of the present invention is to provide a fluorescence on/off probe that uses intramolecular PeT, that is, a novel fluorescent probe capable of carrying out red-colored bio-imaging with high sensitivity by chemical modification of the fluorescein backbone.

### Means Used to Solve the Above-Mentioned Problems

As noted above, the formerly reported Ca²⁺-detecting fluorescent probe CaTM-2-AM has a drawback in that it has poor solubility and does not dissolve in a buffer, and therefore aggregates are formed, resulting in poor sensitivity of cell imaging. The present inventors achieved the present invention after thoroughgoing research having posited that the sensitivity of cell imaging could be improved by increasing solubility via the introduction of a carboxy group.

In other words, the present invention provides [1] a compound represented by general formula (I) below and a salt thereof.

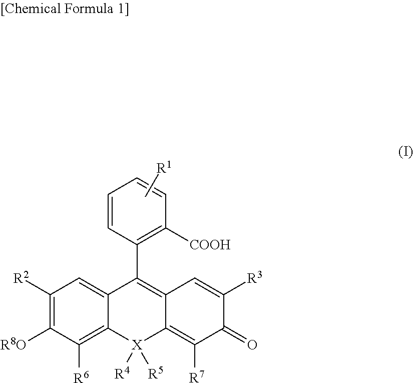

(where:

R¹ represents 1 to 4 monovalent substituents present on a benzene ring, which are the same or different (where at least one of the substituents acts as a capturing group in relation to a substance to be measured);

R² and R³ are, independently, a hydrogen atom, C₁₋₆ alkyl group, or halogen atom;

R⁴ and R⁵ are, independently, a C₁₋₆ alkyl group or aryl group;

R⁶ and R⁷ are, independently, a hydrogen atom, C₁₋₆ alkyl group, or halogen atom;

R⁸ is a substituent acting as a capturing group in relation to the substance to be measured, a hydrogen atom, an alkylcarbonyl group, or an alkylcarbonyloxymethyl group; and

X is a silicon, germanium, or tin atom)

[2] The compound or salt thereof according to [1], wherein X is a silicon or germanium atom.
[3] The compound or salt thereof according to [1] or [2], wherein the capturing group of R¹ is a capturing group for capturing a proton, metal ion, low-oxygen environment, or a reactive oxygen species.
[4] The compound or salt thereof according to any one of [1] to [3], wherein the capturing group of R¹ is a capturing group for capturing a calcium ion.
[5] The compound or salt thereof according to any one of [1] to [4], wherein the capturing group of R¹ binds to a benzene ring via a spacer.
[6] The compound or salt thereof according to [1] represented by general formula (Ia) below.

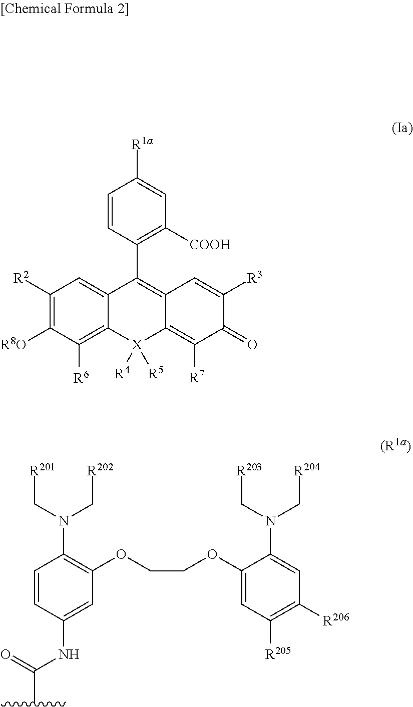

(where:

R² to R⁸ and X are as defined above;

R²⁰¹, R²⁰², R²⁰³, and R²⁰⁴ are a carboxy group or an alkanoyloxyalkyloxycarbonyl group; and

R²⁰⁵ and R²⁰⁶ are, independently, a hydrogen atom, a C₁₋₆ alkyl group, a nitro group, or halogen atom)

[7] The compound or salt thereof according to [6] represented by general formula (Ib) below.

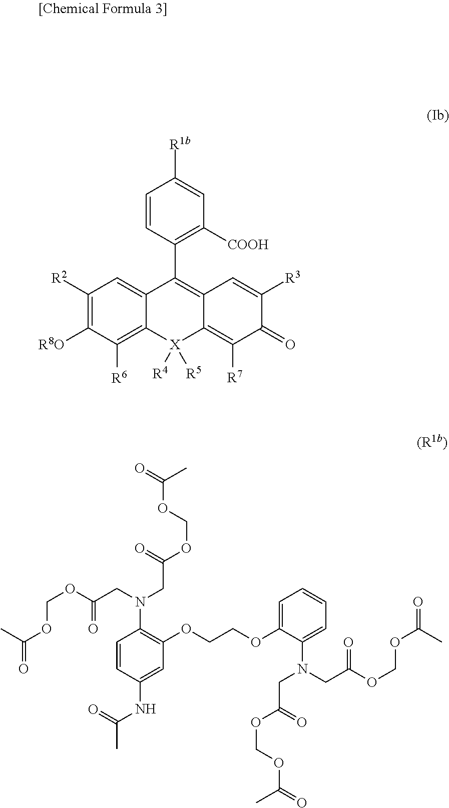

(where R² to R⁸ and X are as defined above)

[8] The compound or salt thereof according to any one of [1] to [7], wherein the capturing group of R⁸ is a capturing group for capturing a proton, a reactive oxygen species, and a glycohydrolase.
[9] A fluorescent probe comprising the compound or salt thereof according to any one of [1] to [8].
[10] A method for preparing a compound of formula (I), comprising:

(a) a step for reacting 4-bromoisophthalic acid and t-butyl alcohol to obtain di-tert-butyl 4-bromoisophthalate; and

(b) a step for reacting di-tert-butyl 4-bromoisophthalate and sec-butyl lithium, adding a compound represented by formula (II) below immediately thereafter, and then adding an acid to obtain a compound of formula (III).

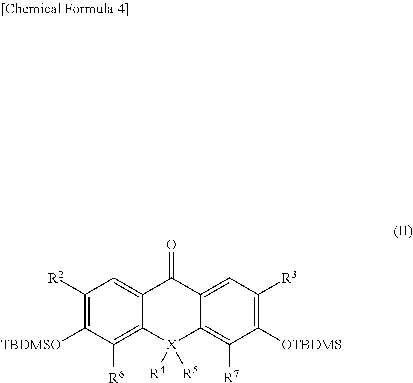

(where R² to R⁷ and X are as defined above)

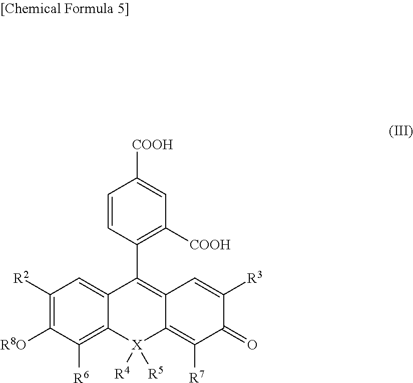

(where R² to R⁸ and X are as defined above)
[11] A method for measuring a substance to be measured, comprising steps of: (a) bringing the substance to be measured into contact with the compound or salt thereof according to any one of [1] to [8]; and (b) measuring fluorescence intensity of the compound after capture of the substance to be measured generated in step (a).

### Advantages of the Invention

The compound represented by general formula (I) below and a salt thereof as provided by the present invention is essentially non-fluorescent prior to capturing a substance to be measured, yields a compound that emits high-intensity red fluorescence by intramolecular PeT after the substance to be measured has been captured, and is highly soluble, thus offering utility as a fluorescent probe capable of measuring pH, metal ions, and reactive oxygen species with high sensitivity.

The compound represented by general formulas (Ia) and (Ib) or salt thereof as provided by the present invention is highly soluble and readily taken into a cell, thus offering utility as a fluorescent probe capable of measuring calcium ions with high sensitivity. Furthermore, since a fluorescent probe capable of measuring calcium ions using a long wavelength can be provided by the compound represented by general formulas (Ia) and (Ib), or salt thereof, as in the present invention, the fluorescent probe can be used in combination with a blue fluorescent probe or the like, and it is possible to track dynamic changes of a cell.

### BRIEF DESCRIPTION OF THE DRAWINGS

FIG. 1 Absorption spectra and fluorescence spectra of ZnTM-1 depending on pHs.

FIG. 2 Absorption spectra and fluorescence spectra of ZnTm-1 upon addition of Zn²⁺.

FIG. 3 Results of cell imaging using ZnTM-1.

FIG. 4 Average fluorescence intensity in each region of interest (ROI) in each cell image of FIG. 3.

FIG. 5 Results of comparison of the fluorescence intensities of CaTM-3-AM and CaTM-2-AM.

FIG. 6 Results of Ca²⁺ during stimulation by histamine and ionomycin using cells to which CaTM-3-AM has been added.

FIG. 7 Fluorescence images at the time points a, b, and c in FIG. 6.

### BEST MODE FOR CARRYING OUT THE INVENTION

Informal notations of compound names in the specification will be described. Descriptions herein are used for facilitating understanding of the descriptive content of the specification, there is no intention to exclude exceptions, and there is no priority given to individual definitions in the present specification.

“TokyoMagenta” refers to a compound that does not have a carboxy group at position 2 of a benzene ring in general formula (I), where R¹ is a hydrogen atom, R² and R³ are hydrogen atoms, R⁴ and R⁵ are a methyl group, R⁶ and R⁷ are hydrogen atoms, R⁸ is a hydrogen atom, and X is a silicon atom. This compound may be abbreviated as “TM.”

In the present specification, unless otherwise stated, “alkyl group” or the alkyl moiety of the substituent group (e.g., alkoxy group or the like) containing the alkyl moiety refers to an alkyl group composed of, e.g., a C₁₋₆, preferably C₁₋₄, and more preferably C₁₋₃ straight, branched, or cyclic configuration, or a combination thereof. More specific examples of the alkyl group include a methyl group, ethyl group, n-propyl group, isopropyl group, cyclopropyl group, n-butyl group, sec-butyl group, isobutyl group, tert-butyl group, cyclopropylmethyl group, n-pentyl group, and n-hexyl group. In the present specification, “halogen atom” may refer to a fluorine atom, chlorine atom, bromine atom, or iodine atom, and is preferably a fluorine atom, chlorine atom, or bromine atom.

In the compound represented by general formula (I), R¹ represents 1 to 4 monovalent substituents present on a benzene ring, which are the same or different and at least one of the substituents acts as a capturing group in relation to a substance to be measured). The substituent group acting as a capturing group may be a substituent group that acts alone as the capturing group, or may be a substituent group that acts as a capturing group by combination with two or more substituent groups on a benzene ring, or preferably by combination with two substituent groups adjacent to each other on a benzene ring. The two substituent groups may bond to form a ring structure, and the ring structure may change to an open ring structure after reaction with a substance to be measured. Alternatively, the two substituent groups may form a ring structure after two adjacent substituent groups react with a substance to be measured. The benzene ring may form a portion of the capturing group in order to act as a capturing group. Furthermore, two or more substituent groups acting alone as a capturing group may bind on the benzene ring, or two or more different types of substituent groups that each act as a capturing group in relation to different substances to be measured may be present on the benzene ring. The substitution position of the single substituent group or two or more substituent groups acting as a capturing group on the benzene ring is not particularly limited, and substitution may occur in any position. R¹ may be solely a capturing group in relation to a substance to be measured and another substituent group except the capturing group may not be present on the benzene ring. Also, the structure resulting from the combination of R¹ and the benzene ring to which R¹ binds may function as a capturing group, and such embodiments are also included in the scope of the present invention.

The type of the substance to be measured is not particularly limited; examples include metal ions (e.g., sodium ions, lithium ions, and other alkali metal ions; and calcium ions and other alkaline-earth metal ions; magnesium ions, zinc ions, and the like), nonmetal ions (carbonate ions, hydroxy ions, and the like), reactive oxygen species (e.g., hydroxyl radical, peroxynitrite, hypochlorite, hydrogen peroxide, and the like), and enzymes. In the present invention, the type of the substance to be measured is preferably metal ions, and more preferably calcium ions.

Various capturing groups for specifically capturing a substance to be measured have been proposed and the capturing group can be selected, as appropriate, in accordance with the type of the substance. Examples that may be used include those described in Japanese Laid-open Patent Application No. 10-226688, International Publication WO 99/51586, Japanese Laid-open Patent Application No. 2000-239272, and International Publication WO 01/62755, as well as Molecular Probes Handbook, 11^{th} Edition, (catalog published by Molecular Probes, Inc.), Chapter 10 (Enzyme Substrates and Assays), Chapter 17 (Probes for Signal Transduction), Chapter 18 (Probes for Reactive Oxygen Species, Including Nitric Oxide), Chapter 19 (Indicators for Ca2+, Mg2+, Zn2+, and Other Metal Ions), Chapter 20 (pH Indicators), and Chapter 21 (Indicators for Na+, K+, Cl—, and Miscellaneous Ions). Naturally, the capturing group is not limited to those listed in the above-noted publications.

The term “capture” in the present specification must not be interpreted with limitation in any meaning; it must be interpreted with the broadest meaning, including the case in which the capturing group captures a metal ion or the like by chelation or the like essentially without bringing about a chemical change, the case in which the chemical structure changes by chemical reaction with the substance to be measured, and the case in which the capturing group is cleaved and isolated by contact with an enzyme.

Examples of the capturing group include those expressed by (A) to (J) below, but capturing groups that may be used in the present invention are not limited thereto.

(A) Zinc-Ion-Capturing Group

(A-1)

A substituent represented by

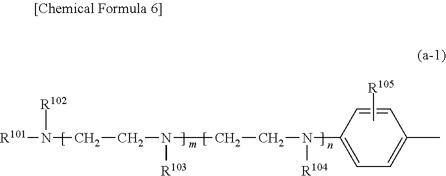

(where R¹⁰¹, R¹⁰², R¹⁰³, and R¹⁰⁴ are independently, a hydrogen atom, alkyl group, 2-pyridylmethyl group, 2-pyridylethyl group, 2-methyl-6-pyridylmethyl group, or 2-methyl-6-pyridylethyl group, provided that at least one group selected from the set consisting of R¹⁰¹, R¹⁰², R¹⁰³, and R¹⁰⁴ represents a group selected from the group consisting of a 2-pyridylmethyl group, 2-pyridylethyl group, 2-methyl-6-pyridylmethyl group, and 2-methyl-6-pyridylethyl group;

R¹⁰⁵ is a hydrogen atom or represents 1 to 4 monovalent substituents present on a benzene ring, which are the same or different;

m and n are, independently, 0 or 1, provided that m and n are not 0 simultaneously).

The above-noted capturing group is disclosed in Japanese Patent No. 4402191 and J. Am. Chem. Soc., 127, pp. 10197-10204, 2005.

A preferred example of the above-noted capturing group is the capturing group represented by the following formula.

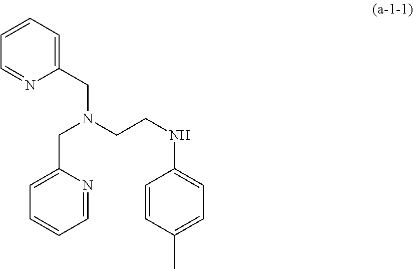 [SMILES: Cc1ccc(NCCN(Cc2ccccn2)Cc2ccccn2)cc1]

Also, these capturing groups may bind to a benzene ring via, e.g., —CO—NH— or another spacer, as described below. For example, the following formula expresses the case in which the capturing group of formula (a-1-1) binds to a benzene ring via —CO—NH— spacer.

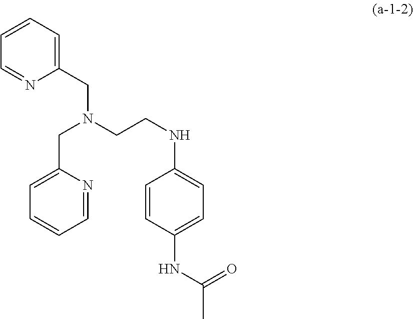 [SMILES: CC(=O)Nc1ccc(NCCN(Cc2ccccn2)Cc2ccccn2)cc1]

(A-2)

A substituent represented by

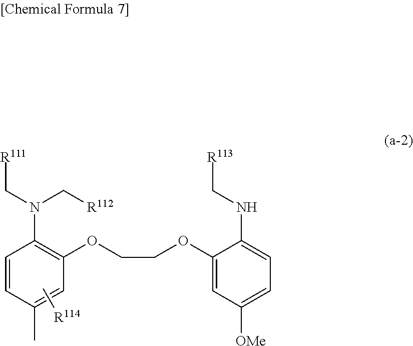

(where R¹¹¹, R¹¹², and R¹³³ are, independently, a carboxy group or salt thereof, R¹¹⁴ is a hydrogen atom or represents 1 to 3 monovalent substituents present on a benzene ring, which are the same or different).

The above-noted capturing group is disclosed in J. Am. Chem. Soc., 124, pp. 776-778, 2002.

(A-3)

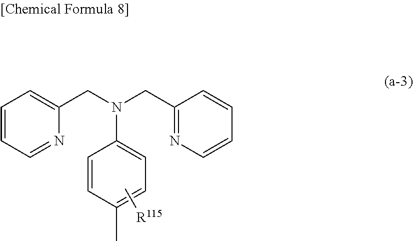

A substituent represented by
(where R¹¹⁵ is a hydrogen atom or 1 to 4 monovalent substituents present on a benzene ring, which are the same or different).

The above-noted capturing group is described in the specification of U.S. Pat. No. 5,648,270.

(A-4)

A substituent represented by

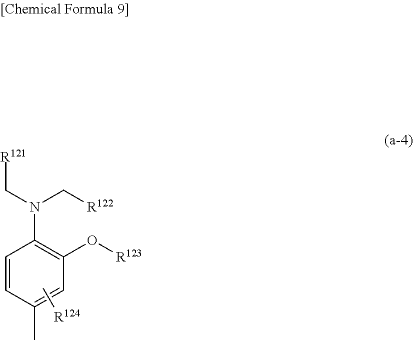

(where R¹²¹ and R¹²² are, independently, a carboxy group or salt thereof, R¹²³ is a C₁₋₆ alkyl group, and R¹²⁴ is 1 to 3 monovalent substituents including a hydrogen atom present on a benzene ring, which are the same or different).

The above-noted capturing group is disclosed in Cell Calcium, 31, pp. 245-251, 2002.

(A-5)

A substituent represented by

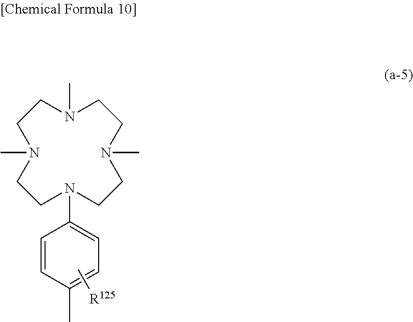

(where R¹²⁵ is a hydrogen atom or represents 1 to 4 monovalent substituents including a hydrogen atom present on a benzene ring, which are the same or different).

The above-noted capturing group is described in Japanese Laid-open Patent Application No. 2000-239272.

(B) Nitrogen-Monoxide-Capturing Group

A substituent represented by

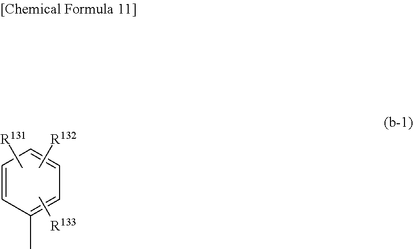

(where R¹³¹ and R¹³² are substituents for substitution in adjacent positions on a benzene ring, and are, independently, an amino group or a C₁₋₆ alkyl mono-substituted amino group, provided that R¹³¹ and R¹³² are not simultaneously a C₁₋₆ alkyl mono-substituted amino group; R¹³³ is a hydrogen atom or represents 1 to 3 monovalent substituents present on a benzene ring, which are the same or different).

The above-noted capturing group is disclosed in Japanese Patent No. 3200024, the specification of U.S. Pat. No. 6,441,197, the specification of U.S. Pat. No. 675,623, and Japanese Patent No. 3967943.

(C) Reactive-Oxygen-Species Capturing Group

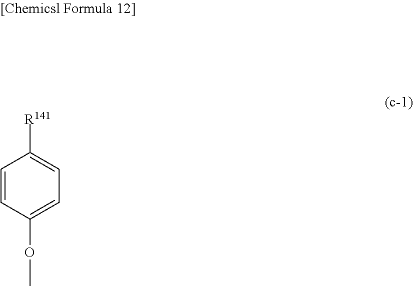

A substituent represented by
(where R¹⁴¹ is an amino group or a hydroxy group).

The above-noted capturing group is disclosed in International Publication WO 2001/064664.

(D) Low-Oxygen-Environment Capturing Group

(D-1)

A substituent represented by

[Chemical Formula 13]

—CO—N(R¹⁵¹)—Y¹—N(R¹⁵²)—X¹—(X²)ᵣ-p-C₅H₄—N═N—Ar—R¹⁵³ (d-1)

(where R¹⁵¹ and R¹⁵² are, independently, a hydrogen atom or C₁₋₆ alkyl group, and R¹⁵¹ and R¹⁵² may bind to each other to form a C₂₋₆ alkylene group; Y¹ is a C₁₋₆ alkylene group; X¹ is a single bond, —CO—, or —SO₂—; X² is —O—Y²—N(R¹⁵⁴)— where Y² is a C₁₋₆ alkylene group, and R¹⁵⁴ is a hydrogen atom or C₁₋₆ alkyl group); r is 0 or 1; p-C₆H₄ is a p-phenylene group; Ar is an aryldiyl group; and R¹⁵³ is a monoalkylamino group or dialkylamino group).

The above-noted capturing group is disclosed in International Publication WO 2010/026743.

(D-2)

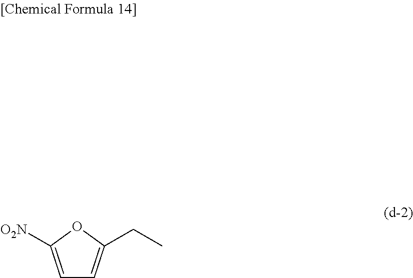

The above-noted capturing group is described in Japanese Laid-open Patent Application No. 2009-275006.

(E) Hydrogen-Peroxide Capturing Group

A substituent represented by

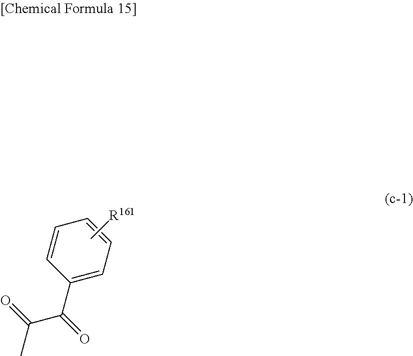

(where R¹⁶¹ is one or more of electron-withdrawing substituents present on a benzene ring)

The above-noted capturing group is disclosed in International Publication WO 2009/110487.

(F) Singlet-Oxygen Capturing Group

A substituent represented by

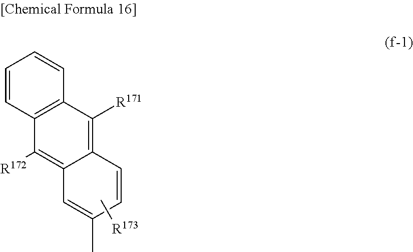

(where R¹⁷¹ and R¹⁷² are, independently, a C₁₋₄ alkyl group or aryl group; R¹⁷³ is a hydrogen atom or represents 1 to 3 monovalent substituents present on a benzene ring, which are the same or different).

The above-noted capturing group is disclosed in Japanese Patent No. 4373608 and International Publication WO 2002/018362.

(G) pH Environment Capturing Group

A substituent represented by

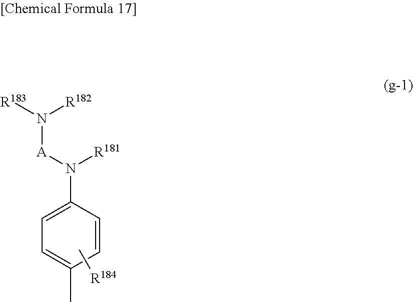

(where R¹⁸¹, R¹⁸², and R¹⁸² are, independently, a hydrogen atom, a C₁₋₆ alkyl group that may have a substituent, or an aryl group that may have a substituent, alternatively, R¹⁸¹ and R¹⁸² bind together to represent a C₁₋₃ alkylene group, or R¹⁸¹ and R¹⁸³ bind together to represent a C₁₋₃ alkylene group; A is a C₁₋₃ alkylene group that may have a substituent; and R¹⁸⁴ is a hydrogen atom or represents 1 to 4 monovalent substituents present on a benzene ring, which are the same or different).

The above-noted capturing group is disclosed in International Publication WO 2008/099914 and International Publication WO 2008/059910.

(H) Magnesium-Ion Capturing Group

A substituent represented by

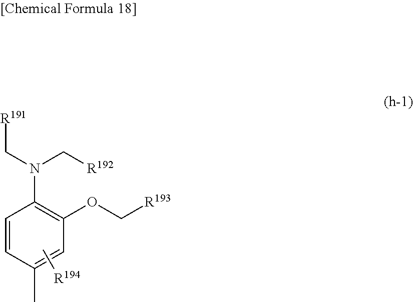

(where R¹⁹¹, R¹⁹², and R¹⁹³ are, independently, a carboxy group or salt thereof; R¹⁹⁴ is a hydrogen atom or 1 to 3 monovalent substituents present on a benzene ring, which are the same or different).

The above-noted capturing group is disclosed in American Journal of Physiology, 256, C540-548, 1989.

(I) Sodium Ion and Potassium Ion Capturing Group

A substituent represented by

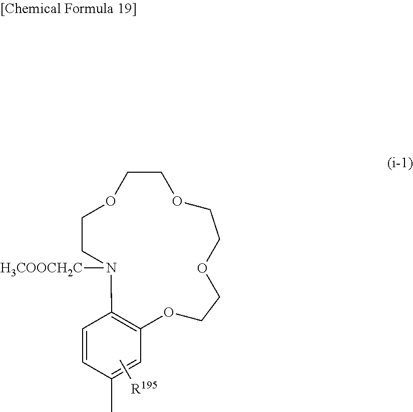

(where R¹⁹⁵ is a hydrogen atom or represents 1 to 3 monovalent substituents present on a benzene ring, which are the same or different).

The above-noted capturing group is disclosed in Bioorg. Med. Chem. Lett., 15, pp. 1851-1855, 2005.

(J) Calcium Ion Capturing Group

A substituent represented by

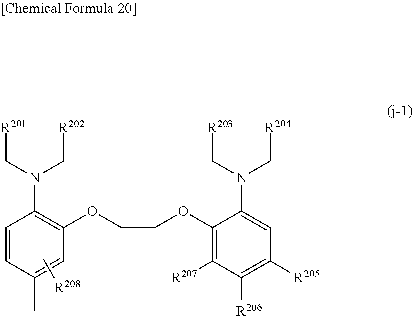

In this case, the capturing group of R¹, or the capturing group formed by a combination of R¹ and the benzene ring to which R¹ binds is a capturing group represented by the formula (j-1) (where R²⁰¹, R²⁰², R²⁰³, and R²⁰⁴ are independently, a carboxy group or salt thereof; R²⁰⁵, R²⁰⁶, and R²⁰⁷ are, independently, a hydrogen atom, halogen atom (fluorine atom, chlorine atom, and bromine atom), C₁₋₆ alkyl group, or nitro group; R²⁰⁸ is a hydrogen atom or represents 1 to 3 monovalent substituents present on a benzene ring, which are the same or different).

These substituents may bind to a benzene ring via, e.g., —CO—NH— or another spacer.

A preferred compound of the present invention in which a calcium-capturing group has been introduced is represented by general formula (Ia) below.

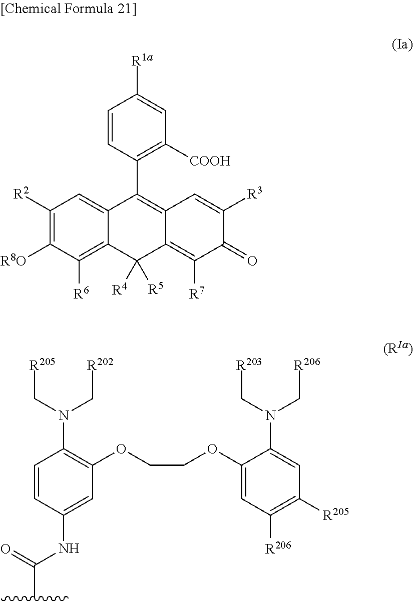

(where R² to R⁸ and X are as defined above;

R²⁰¹, R²⁰², R²⁰³, and R²⁰⁴ are a carboxy group or an alkanoyloxyalkyloxycarbonyl group; and

R²⁰⁵ and R²⁰⁶ are, independently, a hydrogen atom, a C₁₋₆ alkyl group, a nitro group, or halogen atom).

Examples of compounds represented by general formula (Ia) include:

(1) a compound in which R², R³, R⁶, and R⁷ are, independently, a hydrogen atom, fluorine atom, or chlorine atom, R⁴ and R⁵ are, independently, a methyl group, ethyl group, or other C₁₋₆ alkyl group, R⁸ is a hydrogen atom, R²⁰¹, R²⁰², R²⁰³, and R²⁰⁴ are a carboxy group, R²⁰⁵ and R²⁰⁶ are, independently, a hydrogen atom, methyl group or other C₁₋₆ alkyl group, a nitro group, or fluorine atom; and

(2) a compound in which R², R³, R⁶, and R⁷ are, independently, a hydrogen atom, fluorine atom, or chlorine atom, R⁴ and R⁵ are, independently, a methyl group, ethyl group, or other C₁₋₆ alkyl group, R⁸ is a hydrogen atom, acetyl group or other alkanoyl group, or an acetoxymethyl group or other alkanoyloxyalkyl group, R²⁰¹, R²⁰², R²⁰³, and R²⁰⁴ are a acetoxymethyloxycarbonyl group or other alkanoyloxyalkyloxycarbonyl group, R²⁰⁵ and R²⁰⁶ are, independently, a hydrogen atom, methyl group or other C₁₋₆ alkyl group, a nitro group, or fluorine atom;

More preferable examples of compounds represented by general formula (Ia) include those represented by general formula (Ib).

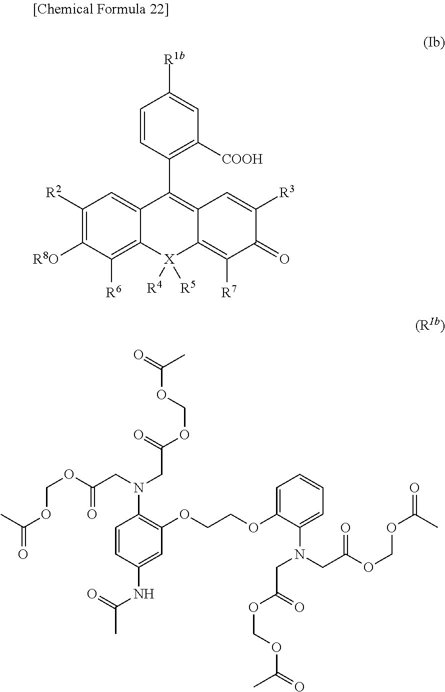

(where R² to R⁸ and X are as defined above)

In formula (Ib), it is preferred that R², R³, R⁶, and R⁷ be, independently, a hydrogen atom, fluorine atom, or chlorine atom, R⁴ and R⁵ be, independently, a methyl group, ethyl group, or other C₁₋₆ alkyl group, R⁸ be a hydrogen atom, acetyl group or other alkanoyl group, or an acetoxymethyl group or other alkanoyloxyalkyl group, and X is preferably a silicon atom or a germanium atom, more preferably a silicon atom.

The above-noted calcium-capturing group is disclosed in Molecular Probes Handbook, 11^{th} Edition, (catalog published by Molecular Probes, Inc.), Chapter 19 (Indicators for Ca2+, Mg2+, Zn2+, and Other Metal Ions), and in J. Biol. Chem., 260, pp. 3440-3450, 1985.

In the capturing group expressed by the above-described (A) to (J), R¹ may be directly substituted in the benzene ring of the compound represented by general formula (I), or may be substituted in the benzene ring of the compound represented by general formula (I) with a suitable spacer interposed therebetween. Examples of spacers that can be used include —CO—NH—, as indicated in formulas (R^{1a}) and (R^{1b}) noted above. In a capturing group having a terminal benzene ring (including those that are polycyclic), e.g., (A), (B), (F), (G), (H), (I), and (J), the terminal benzene ring may include a benzene ring in which R¹ of the compound represented by general formula (I) is substituted.

As regards the capturing groups expressed by (A) to (J) above, all methods disclosed in the above-noted publications and all other disclosures of the above-noted patent publications and reference documents are incorporated by reference in the disclosure of the present specification for the compound represented by general formula (I), or a salt thereof, as provided according to the present invention.

The fluorescence activation characteristics of the compound of the present invention are achieved by intramolecular photoinduced electron transfer (PeT) without limiting to any particular theory (PeT is disclosed in detail in J. Am. Chem. Soc., 125, 8666-8671, 2003; J. Am. Chem. Soc., 127, 4888-4894, 2005; J. Am. Chem. Soc., 128, 10640-10641, 2006; J. Am. Chem. Soc., 126, 14079-14085, 2004; and Yakugaku Zasshi, 126, 901-913, 2006.). PeT is one method of fluorescence quenching and includes a phenomenon in which a singlet excited fluorophore generated by irradiation of excitation light emits fluorescence and electron transfer occurs from an adjacent electron donor site (PeT donor) more quickly than the speed involved in returning to a normal state whereby fluorescence quenching occurs (a-PeT), and a phenomenon in which a singlet excited fluorophore generated by irradiation of excitation light emits fluorescence and electron transfer occurs to an adjacent electron acceptor site (PeT acceptor) more quickly than the speed involved in returning to a normal state, whereby fluorescence quenching occurs (d-PeT). The specific details of the oxidation potential of the capturing group and/or the benzene ring to which R¹ binds can be readily acquired by, e.g., computing the oxidation potential of the capturing group and/or the benzene ring in accordance with quantum chemistry techniques. Reduced oxidation potential of the capturing group and/or benzene ring indicates that the electron density of the capturing group and/or the benzene ring is increased, and this corresponds to an increased highest occupied molecular orbital (HOMO) energy. For example, the HOMO energy of the capturing group site and/or the benzene ring site can be determined by the density functional method (B3LYP/6-31G(d)). When R¹ includes a capturing group for a substance to be measured, it is necessary to select: a capturing group in which there is a change in the oxidation potential of the capturing group site itself and/or the benzene ring to which R¹ binds after the substance to be measured has been captured; or a capturing group that has a group having an oxidation potential in which the compound represented by general formula (I) is essentially non-fluorescent before the capturing group site itself captures the substance to be measured and the group is cleaved and isolated when the substance to be measured is captured.

In the compound of the present invention represented by general formula (I), when R¹ acting as a capturing group changes the oxidation potential of the benzene ring to which R¹ binds, the R¹ acting as a capturing group or, in the case that a R¹ other than the capturing group is present, a combination of the R¹ and the R¹ acting as a capturing group is selected so that, e.g., (1) the oxidation potential of the benzene ring to which R¹ binds is 1.57 V or less, preferably 1.26 V or less so that the compound represented by general formula (I) is essentially non-fluorescent before a substance to be measured is captured, and (2) after a substance to be measured is captured, the oxidation potential of the benzene ring to which R¹ binds is 1.75 V or more, preferably 1.98 V or more, so that the post-capture compound derived from the compound represented by general formula (I) is essentially highly fluorescent. Also, in the compound of the present invention represented by general formula (I), when the R¹ acting as a capturing group essentially does not affect the oxidation potential of the benzene ring to which R¹ binds after a substance to be measured is captured and the capture of the substance is detected by a change in the oxidation potential of the capturing group itself, the groups must be selected as a combination in which the oxidation potential of the benzene ring to which R¹ binds is, e.g., 1.75 V or more, preferably 1.98 V or more so that the oxidation potential of the benzene ring to which R¹ binds does not affect fluorescence activation; i.e., so that the compound obtained after capture by the capturing group has occurred is essentially highly fluorescent.

In the compound of the present invention represented by general formula (I), when the R¹ acting as a capturing group is a group in which the capturing group itself has essentially a low oxidation potential so that the compound represented by general formula (I) is essentially non-fluorescent, the groups must be selected as a combination in which e.g., (1) the oxidation potential of the group having an essentially low oxidation potential is 1.57 V or less, preferably 1.26 V or less, and (2) the oxidation potential of the group having an essentially low oxidation potential increases to 1.75 V or more, preferably 1.98 V or more so that, the compound resulting from the capture derived from the compound represented by general formula (I) is essentially highly fluorescent due to the subject substance being captured.

In the compound of the present invention represented by general formula (I), the above-noted theory also applies to the case in which R⁸ acts as a capturing group.

Japanese Laid-open Patent Application No. 2012-032373 indicates that a compound or salt thereof in which R⁸ of general formula (I) of the present invention is not a hydrogen atom has a property in which R⁸ becomes a hydrogen atom by contact with a substance to be measured and thereby changes into a compound which has an opened intramolecular spirolactone ring and emits an intense red fluorescence, and therefore, the compound or salt thereof of the above-noted publication is useful as a mother-nucleus compound for manufacturing a fluorescent probe capable of measuring reactive oxygen species, various enzymes, and the like with high sensitivity through the use of this property. Consequently, combining the above-noted property of the compound represented by general formula (I), or a salt thereof, as provided by the present invention and R¹ in the compound represented by general formula (I), or a salt thereof, as provided by the present invention makes it possible to obtain a fluorescent probe in which the fluorescence characteristics change considerably only when a plural substances to be measured have been captured. For example, a monovalent group cleaved by a β-galactosidase can be introduced to R⁸ in the compound represented by general formula (I), or a salt thereof, and a group for capturing calcium ions can be introduced to R¹ to measure excitation light near 580 nm, thus allowing the compound represented by general formula (I), or a salt thereof, as provided by the present invention to be used as a fluorescent probe for remaining non-fluorescent when β-galactosidase or calcium ions are present alone, and emitting fluorescence only when β-galactosidase and calcium ions are simultaneously present.

In order to bind the above-described fluorescent compound and the compound represented by general formula (I), or a salt thereof, as provided by the present invention, it is possible to introduce a group for binding to the substituent group which is substituted by the compound represented by general formula (I), or salt thereof, as provided according to the present invention. Examples of such a group include an amino group, carboxy group and active ester groups thereof (succinimidyl ester or the like), formyl group, hydroxy group, mercapto group, maleimide group, isothiocyanate group, and isocyanate group.

Among the substituent groups represented by R¹ present on a benzene ring in the compound represented by general formula (I), substituent groups other than those acting as a capturing group can also be substituted in any position on the benzene ring. There may also be cases in which substituent groups other than those acting as a capturing group be advantageously absent on the benzene ring. When a substituent group other than those acting as a capturing group is present on the benzene ring, it is preferred that one or two of such substituent groups be present. When a substituent group other than those acting as a capturing group is present on the benzene ring, the substituent group can be substituted in any position on the benzene ring.

Though there is no particular limitation to the type of the monovalent substituent group other than that acting as a capturing group among the substituent groups represented by R¹; preferred examples may be selected from the group consisting of a C₁₋₆ alkyl group, a C₁₋₆ alkenyl group, a C₁₋₆ alkynyl group, a C₁₋₆ alkoxy group, a hydroxy group, a carboxy group, a sulfonyl group, an alkoxycarbonyl group, a halogen atom, and an amino group. These monovalent substituent groups may further have one or more any substituent groups. For example, one or more halogen atoms, carboxy groups, sulfonyl groups, hydroxy groups, amino groups, alkoxy groups, or the like may be present in the alkyl group represented by R¹, and, for example, the alkyl group represented by R¹ may be an alkyl halide group, a hydroxyalkyl group, a carboxyalkyl group, an aminoalkyl group, or the like. Also, one or two alkyl groups may be present in the amino group represented by R¹, and the amino group represented by R¹ may be a monoalkyl amino group or a dialkyl amino group. Furthermore, in the case that the alkoxy group represented by R¹ has a substituent group, examples thereof include a carboxy-substituted alkoxy group and an alkoxycarbonyl-substituted alkoxy group, and more specific examples include a 4-carboxybutoxy group and a 4-acetoxymethyloxycarbonylbutoxy group.

Among the monovalent substituent groups represented by R¹, when two substituent groups other than those acting as a capturing group are present on the benzene ring, the two substituent groups are preferably selected from the group consisting of, e.g., a C₁₋₆ alkyl group, a C₁₋₆ alkoxy group, and a carboxy group, and are more preferably selected from the group consisting of a C₁₋₆ alkyl group and a C₁₋₆ alkoxy group. The alkoxy group (e.g., an unsubstituted alkoxy group, a monocarboxy-substituted alkoxy group, a monoalkoxycarbonyl-substituted alkoxy group, and a 4-acetoxymethyloxycarbonylbutoxy group) is preferably present in another position on the benzene ring.

R² and R³ independently represent a hydrogen atom, a C₁₋₆ alkyl group, or a halogen atom. When R² or R³ represents an alkyl group, one or more halogen atoms, carboxy groups, sulfonyl groups, hydroxy groups, amino groups, alkoxy groups, or the like may be present in the alkyl group, and, for example, the alkyl group represented by R² and R³ may be an alkyl halide group, a hydroxyalkyl group, a carboxyalkyl group, or the like. R² and R³ are, independently, preferably a hydrogen atom or a halogen atom, and it is more desirable that R² and R³ both be hydrogen atoms, or that R² and R³ both be fluorine atoms or chlorine atoms.

R⁴ and R⁵ independently represent a C₁₋₆ alkyl group or aryl group. R⁴ and R⁵ are, independently, preferably a C₁₋₃ alkyl group, and R⁴ and R⁵ are both more preferably a methyl group. One or more halogen groups, carboxy groups, sulfonyl groups, hydroxy groups, amino groups, alkoxy groups, or the like may be present in the alkyl group represented by R⁴ and R⁵, and the alkyl group represented by R⁴ and R⁵ may be, e.g., an alkyl halide group, a hydroxyalkyl group, a carboxyalkyl group, or the like. When R⁴ and R⁵ represent an aryl group, the aryl group may be a monocyclic aromatic group or condensed aromatic group, and the aryl ring may include one or more ring-structured heteroatoms (e.g., nitrogen atom, sulfur atom, oxygen atom, or the like). A phenyl group is preferred as the aryl group. One or more substituent groups may be present on the aryl ring. One or more substituent groups, e.g., a halogen atom, carboxy group, sulfonyl group, hydroxy group, amino group, alkoxy group, or the like may be present.

R⁶ and R⁷ are, independently, a hydrogen atom, C₁₋₆ alkyl group, or halogen atom, to which the explanations provided for R² and R³ are also applied. X is a silicon atom, germanium atom, or tin atom, and is preferably a silicon atom.

R⁸ is a substituent acting as a capturing group in relation to a substance to be measured, a hydrogen atom, an alkylcarbonyl group, or an alkylcarbonyloxymethyl group. Examples of the alkylcarbonyl group that may be used include alkylcarbonyl groups having about 1 to 13 carbon atoms, preferably about 1 to 7 carbon atoms, more preferably about 1 to 5 carbon atoms. The same applies to the alkylcarbonyl group in an alkylcarbonyloxymethyl group. For example, an acetoxymethyl group or the like can be advantageously used. In the compound represented by general formula (I), the lipid solubility of the compound represented by general formula (I) is increased by a compound in which R⁸ is an alkylcarbonyl group or an alkylcarbonyloxymethyl group, and the compound readily passes through a cell membrane and is taken into a cell. Therefore, a compound in which R⁸ is an alkylcarbonyl group or an alkylcarbonyloxymethyl group can be advantageously used when a substance to be measured in a cell will be measured using a bio-imaging technique.

A compound represented by the general formulas (I), (Ia), and (Ib) may be present as a salt. Examples of a salt include a base-addition salt, an acid-addition salt, and an amino acid salt. Examples of the base-addition salt include salts of sodium, potassium, calcium, magnesium, and other metals; ammonium; and triethyl amine, piperidine, morpholine, and other organic amine salts. Examples of the acid-addition salt include hydrochlorides, sulfates, nitrates, and other mineral acid salts; and methanesulfonates, p-toluenesulfonates, citrates, oxalates, and other organic acid salts. An example of the amino acid salt is glycine salt. As shall be apparent, salts of the compound of the present invention are not limited to the foregoing.

A compound represented by the general formulas (I), (Ia), and (Ib) may have one or more asymmetric carbons in accordance with the substituent species, and an optical isomer, or diastereoisomer or other stereoisomer may be present. An unadultered stereoisomer, any mixture of stereoisomers, racemates, and the like are also included in the scope of the present invention. The compound of the present invention represented by general formulas (I), (Ia), and (Ib), or salt thereof, may also be present as a hydrate or solvate, and all of these substances are included in the scope of the present invention. The type of solvent that forms the solvate is not particularly limited; examples include ethanol, acetone, isopropanol, and other solvents.

Method for Synthesizing the Compound of the Present Invention

The compound of general formula (I) of the present invention can be synthesized using a method including steps (a) and (b) in the synthesizing scheme described below. In the following description, the compound of general formula (Ib) of the present invention will be used as an example, but other compounds can also be synthesized using the same method.

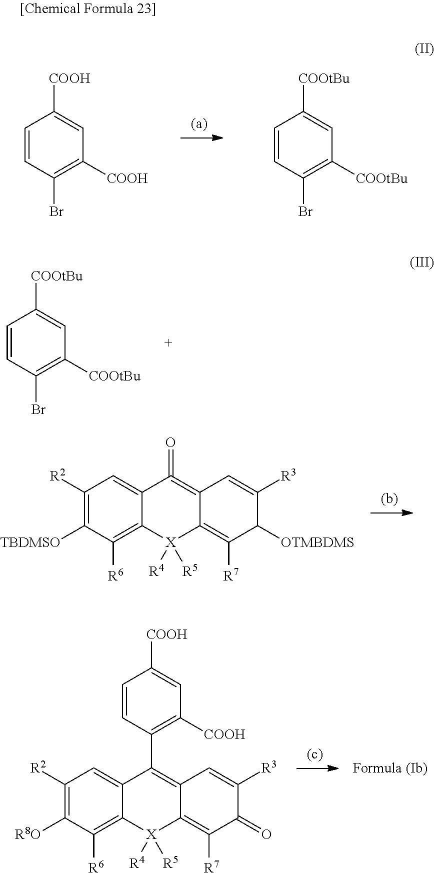

In formulas (II) and (III), R² to R⁸ and X are as defined by formula (I). TBDMS in formula (II) refers to a tert-butyldimethylsilyl group.

(1) Step (a)

Di-tert-butyl 4-bromoisophthalate can be synthesized by adding 4-bromoisophthalic acid to dichloromethane or another solvent together with DCC, DMAP and stirring, then adding dichloromethane in which tBuOH has been dissolved, and stirring the system at room temperature for about one night.

(2) Step (b)

The compound represented by general formula (III) can be synthesized by adding di-tert-butyl 4-bromoisophthalate obtained in step (a) and dehydrated tetrahydrofuran (THF) to a dry, argon-substituted flask, cooling the system to −78° C., dropping 0.5 to 0.7 equivalent sec-BuLi, immediately thereafter (preferably 10 to 30 seconds later) adding the resultant obtained by dissolving the compound represented by general formula (II) in 5 mL of dehydrated THF, restoring the system to room temperature, stirring the system for 1 hour at room temperature, adding and stirring HCl or other acid, extracting the resultant using dichloromethane or other solvent, drying the system, and thereafter adding 5 mL of TFA to the residue and stirring the system for 1 hour.

The compound represented by general formula (II) can be synthesized with reference to International Application WO 2012/111817.

(3) Step (c)

The compound represented by general formula (Ib) can be obtained by dissolving the compound of general formula (III) obtained in step (b), 2 eq of 5-amino-BAPTA-tetraacetoxymethyl ester, 2.5 eq of HATU, and 2.5 eq of HOBt in DMF or another solvent, and stirring the system for about one night at room temperature.

The compound represented by general formulas (I) and (Ia) can be similarly synthesized by using, in lieu of 5-amino-BAPTA-tetraacetoxymethyl ester, a compound (e.g., N,N-bis(2-pyridinylmethyl)-1,4-benzendiamine) that would yield a capturing group expressed by (A) to (J) above.

A compound or salt thereof represented by the general formulas (I), (Ia), or (Ib) is essentially non-fluorescent before a substance to be measured is captured, whereas it has a property in which highly intense red fluorescence is emitted after the substance to be measured has been captured, and can therefore be used as a fluorescent probe for measuring a substance to be measured. The term “measure” as used in the present specification includes measuring, testing, detecting, and the like carried out for the purpose of quantification, qualification, diagnosis, or the like; and the broadest interpretation must be used.

The method for measuring a substance to be measured using the fluorescent probe of the present invention includes, in general:

a step (a) of bringing a substance to be measured into contact with the compound or salt represented by the general formulas (I), (Ia), and (Ib) to cause the substance to be captured by a capturing group (or R^{1a}, R^{1b}) of R¹, and/or the capturing group of R⁸, and

a step (b) of measuring the fluorescence intensity of the compound generated in step (a) above (corresponding to a compound resulting from chelated bond by a metal ion to a capturing group of R¹; a compound in which the chemical structure changes after a substance to be measured has been captured, e.g., a ring structure is formed, the ring is opened, or another chemical modification has occurred; and/or a compound in which R⁸ is cleaved following the capture of the substance to be measured, or undergoes other chemical modifications). For example, the fluorescent probe or salt thereof of the present invention is dissolved in a physiological salt solution, buffer solution, or other aqueous medium, or in a mixture of an aqueous solution and ethanol, acetone, ethylene glycol, dimethysulfoxide, dimethylformamide, or other water-miscible organic solvent. This solution is added to a suitable buffer containing cells or tissue, and the fluorescence spectrum before and after contact with the substance to be measured can be measured.

The fluorescence of the compound after the substance to be measured has been captured can be measured using an ordinary method, and it is possible to use a method for measuring the fluorescence spectrum in vitro, a method for measuring the fluorescence spectrum in vivo using a bio-imaging technique, or another method. For example, when quantification is to be carried out, it is preferred that a calibration curve be created in advance in accordance with an ordinary method. For example, the fluorescence can be measured at a fluorescence wavelength of about 598 nm with the excitation wavelength at about 582 nm.

The fluorescent probe of the present invention may be used as a composition in a blend with additives ordinarily used in reagent preparations in accordance with requirements. Examples of additives for using reagents in a physiological environment include dissolution aids, pH regulators, buffers, isotonizing agents, and other additives. The blending quantity of these additives can be selected, as appropriate, by a person skilled in the art. These compositions are provided as powder mixtures, freeze-dried substances, granules, tablets, liquid solutions, and other suitable forms.

### EXAMPLES

The present invention is described in more detail below using examples, but the scope of the present invention is not limited by the examples described below.

### Example 1

### (1) Synthesis of (ZnTM-1(5-[[[4-[[2-[bis(2-pyridinylmethyl)amino]ethyl]amino]phenyl]amino]carbonyl]-2-(1,8-difluoro-2,9-dihydro-7-hydroxy-9,9-dimethyl-2-oxo-9-silaanthracen-10-yl)benzoic acid)

A Zn²⁺-detecting fluorescent probe ZnTM-1 was synthesized in accordance with Scheme 1 below.

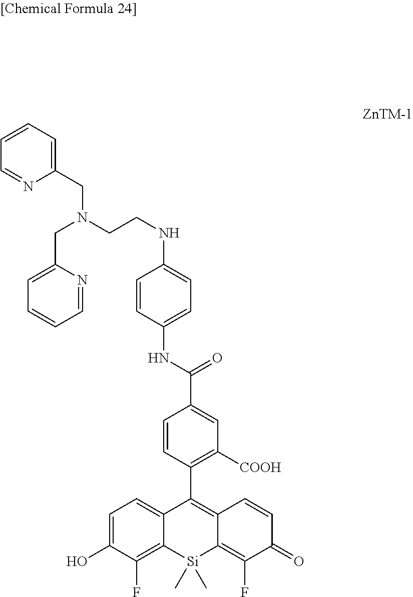 [SMILES: C[Si]1(C)C2=C(F)C(=O)C=CC2=C(c2ccc(C(=O)Nc3ccc(NCCN(Cc4ccccn4)Cc4ccccn4)cc3)cc2C(=O)O)c2ccc(O)c(F)c21]

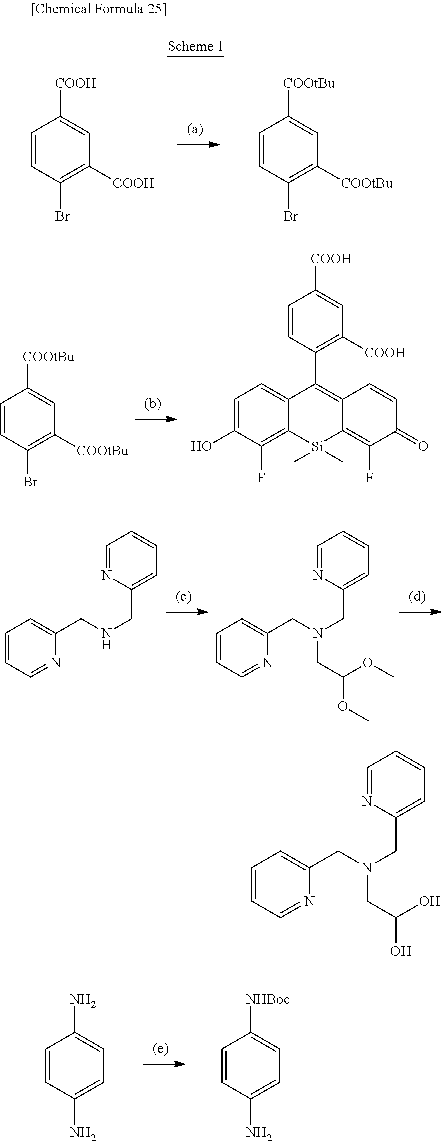

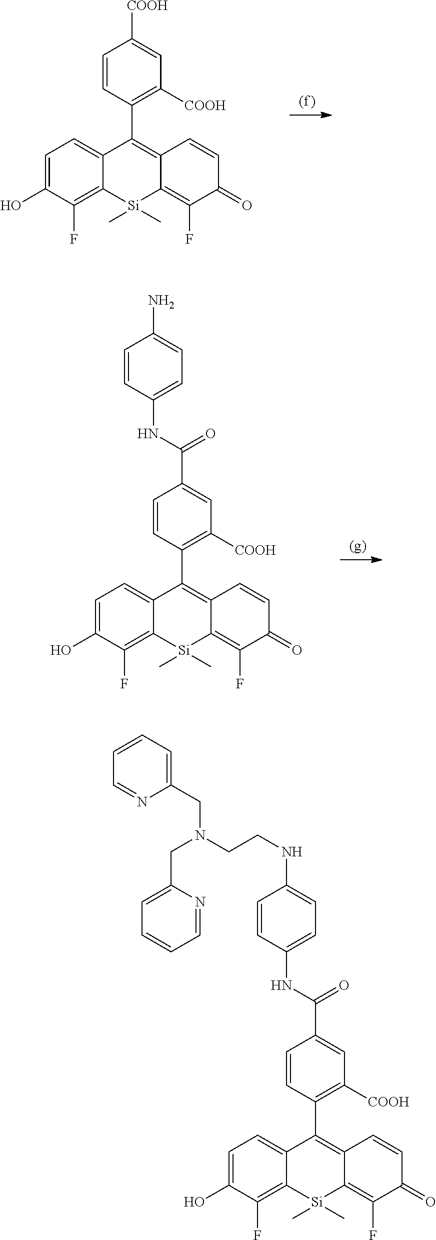 [SMILES: C[Si]1(C)C2=C(F)C(=O)C=CC2=C(c2ccc(C(=O)O)cc2C(=O)O)c2ccc(O)c(F)c21 ; C[Si]1(C)C2=C(F)C(=O)C=CC2=C(c2ccc(C(=O)Nc3ccc(N)cc3)cc2C(=O)O)c2ccc(O)c(F)c21 ; C[Si]1(C)C2=C(F)C(=O)C=CC2=C(c2ccc(C(=O)Nc3ccc(NCCN(Cc4ccccn4)Cc4ccccn4)cc3)cc2C(=O)O)c2ccc(O)c(F)c21]

### Step (a): Synthesis of di-tert-butyl 4-bromoisophthalate

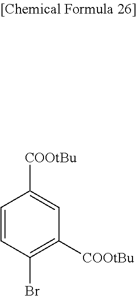

4-Bromoisophthalic acid (6.20 g, 25.3 mmol), DCC (13.1 g, 63.5 mmol), and DMAP (60 mg, 0.49 mmol) were added to dichloromethane (100 mL) and stirred. Dichloromethane (20 mL) in which tBuOH (12 mL) had been dissolved was slowly added thereto at room temperature and the system was stirred for one night. The reaction fluid was washed using a saline solution, the organic layer was then dried using Na₂SO₄ to remove the solvent, and the residue was thereafter refined using column chromatography (silica gel, 4/1 hexane/dichloromethane) to yield di-tert-butyl 4-bromoisophthalate (6.09 g, 17.4 mmol, yield: 67%).

¹H-NMR (300 MHz, CDCl₃): δ 1.59 (s, 9H), 1.62 (s, 9H), 7.66 (d, 1H, J=8.1 Hz), 7.86 (dd, 2H, J=8.1, 2.1 Hz), 8.24 (d, 1H, J=2.1 Hz);

HRMS (ESI⁺): m/z Found 379.0552. calculated 379.0521 for [M+Na]⁺ (3.1 mmu).

### Step (b): Synthesis of 2,4-diCOOH DFTM(4-(1,8-difluoro-2,9-dihydro-7-hydroxy-9,9-dimethyl-2-oxo-9-silaanthracen-10-yl)-1,3-benzenedicarboxylic acid)

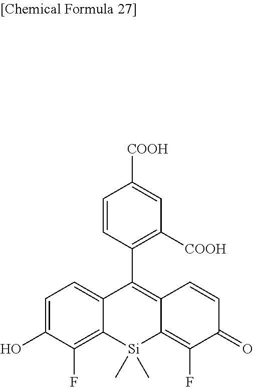 [SMILES: C[Si]1(C)C2=C(F)C(=O)C=CC2=C(c2ccc(C(=O)O)cc2C(=O)O)c2ccc(O)c(F)c21]

Di-tert-butyl 4-bromoisophthalate (357 mg, 1.00 mmol) and dehydrated THF (10 mL) were added to a dry, argon-substituted flask. The system was cooled to −78° C., 1M sec-BuLi (0.6 mmol) was dropped, a mixture obtained by dissolving 4,5-difluoro-3,6-diOTBDMS-Si-xanthone (see International Application WO 2012/111817 for the synthesis method thereof) (10.3 mg, 0.019 mmol) in 5 mL of dehydrated THF was added 30 seconds later, and the system then restored to room temperature and stirred for one hour at room temperature. 10 mL of 2N—HCl was added and the system was stirred for 20 minutes. The resultant was extracted using dichloromethane and washed using a saline solution. The organic layer was dried using Na₂SO₄, the solvent was removed, TFA (2 mL) was added to the residue, and the system was stirred for 1 hour at room temperature. The solvent was removed, and the residue was then partially refined by high-performance liquid chromatography (HPLC) to obtain 2,4-diCOOH DFTM (crude, 3.8 mg, 0.0084 mmol, 44%).

¹H-NMR (300 MHz, CD₃COCD₃): δ 0.69 (s, 3H), 0.82 (s, 3H), 6.81 (d, 2H, J=8.8 Hz), 7.04 (dd, 2H, J=10.3, 8.8 Hz), 7.32 (d, 1H, J=8.1 Hz), 8.31 (dd, 1H, J=8.1, 1.5 Hz), 8.47 (s, 1H) HRMS (ESI⁺): m/z Found 455.0750, calculated 455.0763 for [M+H]⁺ (−1.3 mmu).

### Step (c): Synthesis of N-(2,2-dimethoxyethyl)-N-pyridinylmethyl-2-pyridinmethaneamine

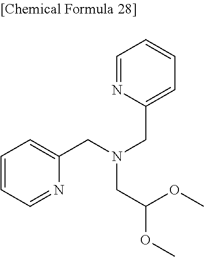 [SMILES: COC(CN(Cc1ccccn1)Cc1ccccn1)OC]

2,2′-Dipicolylamine (1.8 mL, 10.0 mmol) and 2-bromo-1,1-dimethoxyethane (4.8 mL, 40.6 mmol), and Na₂CO₃ (10.0 g, 94.3 mmol) were added to a dry flask, then dissolved in acetonitrile (40 mL), and heated and refluxed for three nights at 80° C. Insoluble matter was filtered out, the solvent was removed, and the residue was refined by column chromatography (silica gel, 19/1 dichloromethane/methanol) to obtain N-(2,2-dimethoxyethyl)-N-pyridinylmethyl-2-pyridinmethaneamine (1.93 g, 6.72 mmol, yield: 67%).

¹H-NMR (300 MHz, CDCl₃): δ 2.79 (d, 2H, J=5.1 Hz), 3.29 (s, 3H), 3.93 (s, 4H), 4.54 (t, 1H, J=5.1 Hz), 7.14 (t, 2H, J=5.9 Hz), 7.56 (d, 2H, J=8.1 Hz), 7.66 (td, 2H, J=7.7, 1.5 Hz), 8.52 (d, 2H, J=4.4 Hz);

¹³C-NMR (75 MHz, CDCl₃): δ 53.5, 55.6, 60.9, 103.6, 121.9, 123.0, 136.3, 148.9, 159.7;

HRMS (ESI⁺): m/z Found 310.1490. calculated 310.1532 for [M+Na]⁺ (−4.2 mmu).

### Step (d): Synthesis of 2-[bis(2-pyridinylmethyl)amino]-1,1-ethanediol

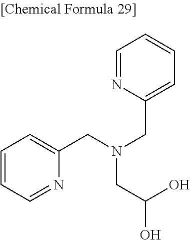 [SMILES: OC(O)CN(Cc1ccccn1)Cc1ccccn1]

30 mL of 1N—HCl was added to N-(2,2-dimethoxyethyl)-N-pyridinylmethyl-2-pyridinmethaneamine (574 mg, 2.00 mmol) and stirred for 7 hours at room temperature. A saturated NaHCO₃ solution was added to form a basic environment, and the resultant was thereafter extracted using dichloromethane and washed with a saline solution. The organic layer was dried using Na₂SO₄ and the solvent was removed to obtain 2-[bis(2-pyridinylmethyl)amino]-1,1-ethanediol (470 mg, 1.95 mmol, yield 95%).

¹H-NMR (400 MHz, CD₃OD): δ 2.73 (t, 2H, J=4.8 Hz), 3.92 (d, 2H, J=2.4 Hz), 4.65 (t, 1H, J=4.8 Hz), 7.26 (dd, 2H, J=6.4, 4.8 Hz), 7.63 (d, 2H, J=8.0 Hz), 7.78 (td, 2H, J=7.6, 1.6 Hz), 8.42 (d, 2H, J=4.8 Hz)

¹³C-NMR (75 MHz, CD₃OD): δ 60.4, 61.8, 98.4, 123.7, 124.9, 138.6, 149.3, 160.7

HRMS (ESI⁺): m/z Found 264.1094. calculated 264.1113 for [M−H₂O+Na]⁺ (−1.9 mmu).

### Step (e): Synthesis of N-Boc-1,4-phenylenediamine

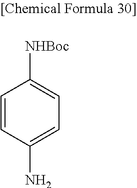 [SMILES: CC(C)(C)OC(=O)Nc1ccc(N)cc1]

1,4-Phenylenediamine (1.15 g, 10.6 mmol) was dissolved in dichloromethane (50 mL), argon was substituted, and the system was cooled to 0° C. and stirred. A solution of di-tert-butyl dicarbonate (463 mg, 2.1 mmol) dissolved in dichloromethane (10 mL) was slowly dropped in the system solution, and the system was stirred for 3.5 hours at 0° C. The solvent was removed and the residue was refined using column chromatography (silica gel, 1/1 ethyl acetate/hexane) to thereby obtain N-Boc-1,4-phenylenediamine (419 mg, 2.01 mmol, yield: 97%).

¹H-NMR (300 MHz, CDCl₃): δ 1.52 (s, 9H), 6.63 (d, 2H, J=8.8 Hz), 7.13 (d, 2H, J=8.8 Hz);

¹³C-NMR (75 MHz, CDCl₃): δ 28.3, 79.9, 115.5, 120.9, 139.6, 142.3, 153.3;

HRMS (ESI⁺): m/z Found 209.1322. calculated 209.1290 for [M+H]⁺ (3.2 mmu).

### Step (f): Synthesis of 2-COOH-4-COPDA DFTM(5-[[4-aminophenyl)amino]carbonyl]-2-(1,8-difluoro-2,9-dihydro-7-hydroxy-9,9-dimethyl-2-oxo-9-silaanthracen-10-yl)-1,3-benzoic acid)

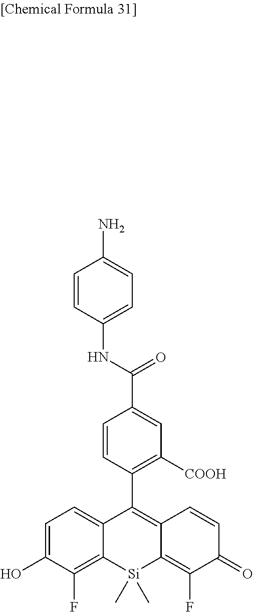 [SMILES: C[Si]1(C)C2=C(F)C(=O)C=CC2=C(c2ccc(C(=O)Nc3ccc(N)cc3)cc2C(=O)O)c2ccc(O)c(F)c21]

2,4-DiCOOH DFTM (3.8 mg, 0.0084 mmol), HATU (15.2 mg, 0.042 mmol), HOBt (6.0 mg, 0.039 mmol), and N-Boc-1,4-phenylenediame (20.8 mg, 0.100 mmol) were dissolved in DMF (1 mL), DIEA (12.9 mg, 0.100 mmol) was added, and the system was stirred for 6 hours at room temperature. AcOEt (20 mL) was added, and the system was washed with 2N—HCl and washed with a saline solution. The organic layer was dried using Na₂SO₄, the solvent was removed, and the residue was refined by HPLC to thereafter obtain 2-COOH-4-COPDA DFTM (2.1 mg, 0.0039 mmol, yield: 46%).

¹H-NMR (300 MHz, CD₃OD): 0.69 (s, 3H), 0.82 (s, 3H), 6.71 (d, 2H, J=8.8 Hz), 6.90 (t, 2H, J=9.2 Hz), 7.23-7.25 (m, 3H), 7.79 (d, 2H, J=8.8 Hz), 8.20 (d, 1H, J=8.1 Hz), 8.47 (s, 1H);

HRMS (ESI %): m/z Found 545.1382. calculated 545.1344 for [M+H]⁺ (3.8 mmu).

### Step (g): Synthesis of ZnTM-1(5-[[[4-[[2-[bis(2-pyridinylmethyl)amino]ethyl]amino]phenyl]amino]carbonyl]-2-(1,8-difluoro-2,9-dihydro-7-hydroxy-9,9-dimethyl-2-oxo-9-silaanthracen-10-yl)benzoic acid)

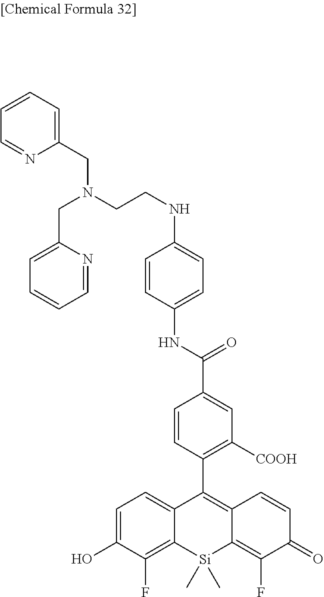 [SMILES: C[Si]1(C)C2=C(F)C(=O)C=CC2=C(c2ccc(C(=O)Nc3ccc(NCCN(Cc4ccccn4)Cc4ccccn4)cc3)cc2C(=O)O)c2ccc(O)c(F)c21]

2-COOH-4-COPDA DFTM (2.1 mg, 0.0039 mmol), 2-[bis(2-pyridinylmethyl)amino]-1,1-ethanediol (9.6 mg, 0.033 mmol), and Na₂SO₄ (10 mg) were dissolved in methanol (3 mL) and the system was stirred for 2 hours at room temperature. NaBH₃CN (2.1 mg, 0.033 mmol) was then added, and the system was stirred for one night at room temperature. A saturated NH₄Cl aqueous solution was added to neutralize the system, and the resultant was thereafter extracted using dichloromethane and washed with a saline solution. The organic layer was dried using Na₂SO₄ and the solvent was removed. The residue was then refined HPLC to obtain ZnTM-1 (0.7 mg, 0.00091 mmol, yield 24%).

¹H-NMR (400 MHz, acetone-d₆): 0.69 (s, 3H), 0.82 (s, 3H), 3.47-3.62 (m, 4H), 4.63 (s, 4H), 6.67 (d, 2H, J=8.8 Hz), 6.79 (d, 2H, J=8.8 Hz), 7.05 (t, 2H, J=9.3 Hz), 7.32 (d, 1H, J=8.3 Hz), 7.44 (t, 2H, J=6.1 Hz), 7.56 (d, 2H, J=8.8 Hz), 7.65 (d, 2H, J=7.3 Hz), 7.92 (td, 2H, J=7.7, 2.1 Hz), 8.28 (d, 1H, J=8.3 Hz), 8.45 (s, 1H), 8.62 (d, 2H, J=3.4 Hz)

HRMS (ESI⁺): m/z Found 770.2591. calculated 770.2610 for [M+H]⁺ (−1.9 mmu).

### (2) Evaluation of the Optical Characteristics of ZnTM-1

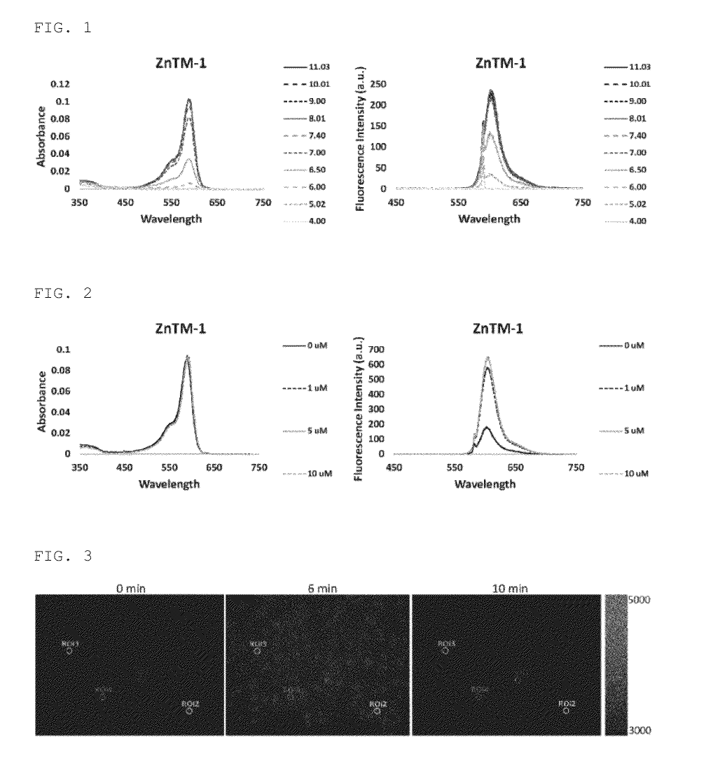

The optical characteristics, absorbance spectra, and fluorescence spectra of the resulting ZnTM-1 depending on pHs are shown in FIG. 1. The absorbance spectra and fluorescence spectra of ZnTM-1 upon addition of Zn²⁺ are shown in FIG. 2.

The pH-induced changes were measured using ZnTM-1 (1 μM) in a buffer of 0.1M sodium phosphate containing 15% DMSO.

The Zn²⁺ addition experiment was carried out by adding ZnSO₄ to a solution obtained by adding ZnTM-1 (1 μM) in a buffer of 0.1M HEPES (pH 7.4) containing 15% DMSO.

pKₐ was determined by biphasic fitting using the absorbance at the absorbance maximum wavelength. EDTA (100 μM) was added in order to eliminate the effect of trace metal ions during the measurement of quantum yield when Zn²⁺ had not been added, and then measurement was carried out. TABLE 1 shows the optical characteristics of ZnTM-1.

[Table 1]

TABLE 1

| | λ_{abs} (nm) | λ_{f1} (nm) | pKₐ | Φ_{f1} |
|---|---|---|---|---|
| ZnTM-1 | 589 | 601 | 7.2, 6.0 | 0.062 |
| ZnTM-1 + 10 eq Zn²⁺ | 589 | 601 | | 0.281 |

### (3) Application of ZnTM-1 to Cell Imaging

Cell imaging was carried out using ZnTM-1. HeLa cells were incubated for 30 minutes at 37° C. together with 10 μM of ZnTM-1 in a Hank's balanced salt solution (HBSS) containing 1% DMSO. The mixture was washed once using HBSS, then HBSS was added and the cells were observed using an epifluorescence microscope IX-71 (Olympus) with an excitation wavelength of 565 to 585 nm and a fluorescence wavelength of 600 to 690 nm. The observation was carried out in every 30 seconds, ZnSO₄ (50 μM) and pyrithione (5 μM), which is an ionophore, were added after 2 minutes and 30 seconds from the beginning of observation, and TPEN (100 μM), which is a cell-membrane-permeating zinc chelator, was added after 6 minutes from the beginning of observation.

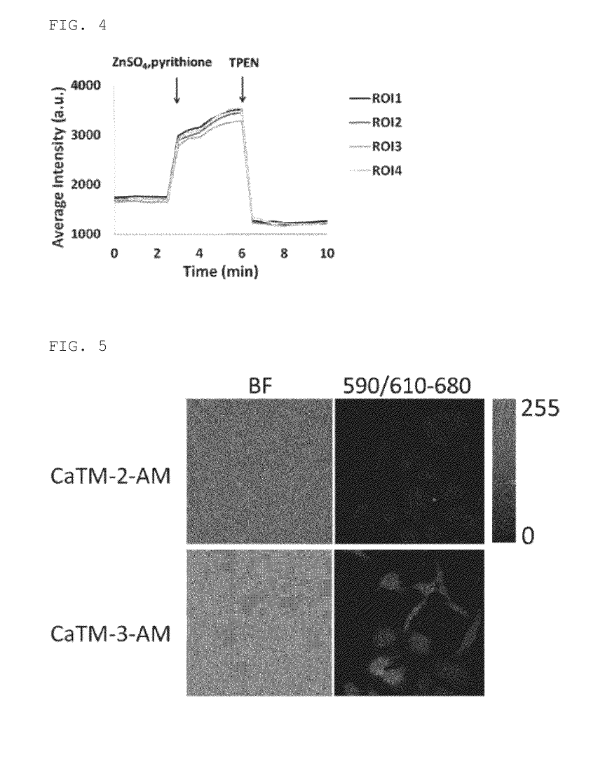

FIG. 3 shows the results of cell imaging. FIG. 4 shows the average fluorescence intensity in each region of interest (ROI) in each cell image of FIG. 3.

FIGS. 3 and 4 demonstrate that ZnTM-1 is taken into the cell and functions effectively as a Zn²⁺-detecting fluorescent probe.

### Experiment 2

### (1) Synthesis of CaTM-3-AM(5-[[[4-[bis[2-[(acetyloxy)methoxy]-2-oxoethyl]amino]-5-[2-[2-[bis[2-[(acetyloxy)methoxy]-2-oxoethyl]amino]phenoxy]ethoxy]-phenyl]amino]carbonyl]-2-(1,8-dichloro-2,9-dihydro-7-hydroxy-9,9-dimethyl-2-oxo-9-silaanthracen-10-yl)-benzoic acid))

A Ca²⁺-detecting fluorescent probe CaTM-3-AM was synthesized in accordance with Scheme 2 below.

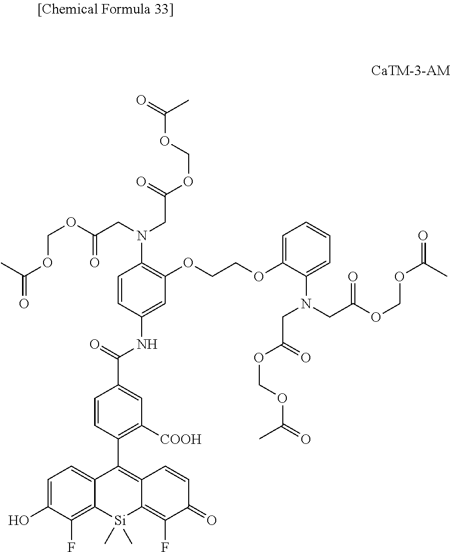 [SMILES: CC(=O)OCOC(=O)CN(CC(=O)OCOC(C)=O)c1ccccc1OCCOc1cc(NC(=O)c2ccc(C3=C4C=CC(=O)C(F)=C4[Si](C)(C)c4c3ccc(O)c4F)c(C(=O)O)c2)ccc1N(CC(=O)OCOC(C)=O)CC(=O)OCOC(C)=O]

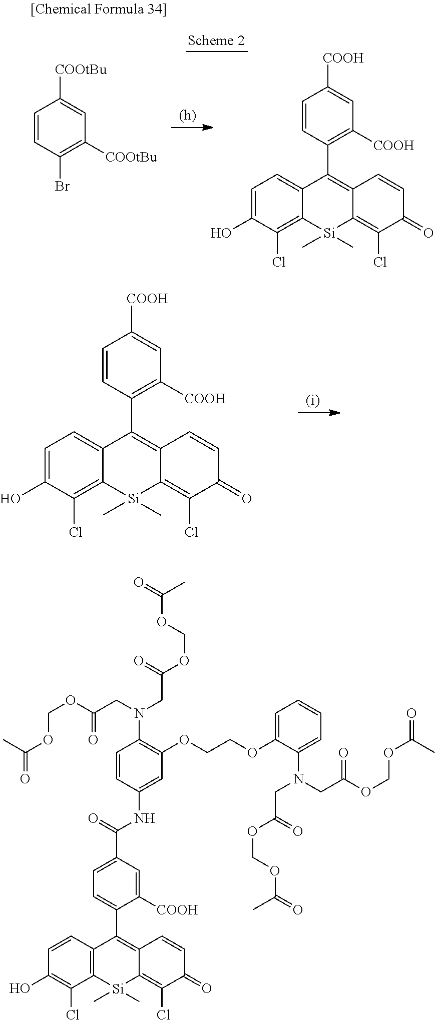

### Step (h): Synthesis of 2,4-diCOOH DCTM(4-(1,8-dichloro-2,9-dihydro-7-hydroxy-9,9-dimethyl-2-oxo-9-silaanthracen-10-yl)-1,3-benzenedicarboxylic acid)

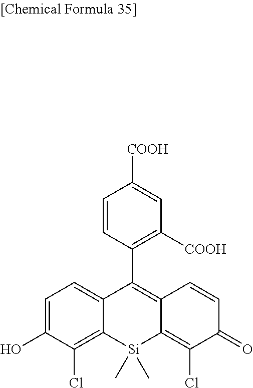 [SMILES: C[Si]1(C)C2=C(Cl)C(=O)C=CC2=C(c2ccc(C(=O)O)cc2C(=O)O)c2ccc(O)c(Cl)c21]

Di-tert-butyl 4-bromoisophthalate (357 mg, 1.00 mmol) and dehydrated THF (5 mL) were added to a dry, argon-substituted flask. The system was cooled to −78° C., 1 M sec-BuLi (0.7 mmol) was dropped, a mixture obtained by dissolving 4,5-dichloro-3,6-diOTBDMS-Si-xanthone (see International Application WO 2012/111817 for the synthesis method thereof) (40.0 mg, 0.0705 mmol) in 5 mL of dehydrated THF was added 10 seconds later, and the system then restored to room temperature and stirred for one hour at room temperature. 10 mL of 2N—HCl was added and the system was stirred for 20 minutes. The resultant was extracted using dichloromethane and washed using a saline solution. The organic layer was dried using Na₂SO₄, the solvent was removed, TFA (5 mL) was added to the residue, and the system was stirred for 1 hour at room temperature. The solvent was removed, and the residue was then refined by HPLC to obtain 2,4-diCOOH DCTM (18.1 mg, 0.0371 mmol, yield: 53%).

¹H-NMR (300 MHz, CD₃OD): δ 0.91 (s, 6H), 6.87 (d, 2H, J=8.8 Hz), 6.91 (d, 2H, J=8.8 Hz), 7.05 (d, 1H, J=8.1 Hz), 8.21 (dd, 1H, J=8.1, 1.5 Hz), 8.49 (d, 1H, J=1.5 Hz)

¹³C-NMR (100 MHz, CD₃OD): δ −0.1, 0.5, 91.4, 119.7, 124.0, 124.9, 127.8, 128.0, 128.3, 133.5, 135.6, 135.9, 137.5, 154.4, 162.3, 167.7, 171.9

HRMS (ESI⁺): m/z Found 487.0210. calculated 487.0171 for [M+H]⁺ (3.9 mmu).

### Step (i): Synthesis of CaTM-3-AM(5-[[[4-[bis[2-[(acetyloxy)methoxy]-2-oxoethyl]amino]-5-[2-[2-[bis[2-[(acetyloxy)methoxy]-2-oxoethyl]amino]phenoxy]ethoxy]-phenyl]amino]carbonyl]-2-(1,8-dichloro-2,9-dihydro-7-hydroxy-9,9-dimethyl-2-oxo-9-silaanthracen-10-yl)-benzoic acid))

2,4-DiCOOH DCTM (9.8 mg, 0.020 mmol), 5-amino-BAPTA-tetraacetoxymethyl ester (31.2 mg, 0.040 mmol), HATU (18.5 mg, 0.050 mmol), and HOBt (7.7 mg, 0.050 mmol) were dissolved in in DMF (1 mL), and the system was stirred for one night at room temperature. The solvent was removed, and the residue was thereafter refined by HPLC to obtain CaTM-3-AM (8.6 mg, 0.0069 mmol, yield: 34%).

¹H-NMR (300 MHz, CD₃OD): δ 0.84 (s, 3H), 1.00 (s, 3H), 2.00 (s, 6H), 2.02 (s, 6H), 4.17 (s, 8H), 4.30 (s, 4H), 5.58 (s, 4H), 5.59 (s, 4H), 6.83-7.09 (m, 9H), 7.08 (d, 1H, J=8.1 Hz), 7.17 (dd, 1H, J=8.8, 2.2 Hz), 7.46 (d, 1H, J=2.2 Hz), 8.11 (dd, 1H, J=8.1, 1.5 Hz), 8.44 (s, 1H);

HRMS (ESI⁺): m/z Found 1248.2420. calculated 1428.2451 for [M+H]⁺ (−3.1 mmu).

### (2) Application of CaTM-3-AM to Cell Imaging

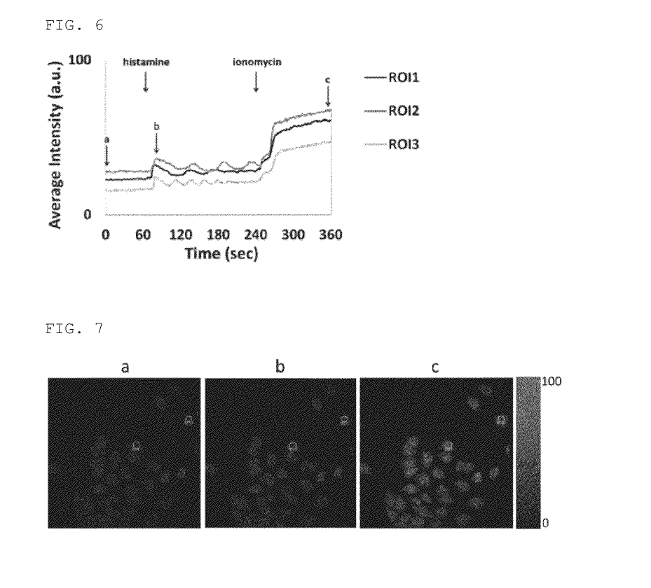

Ca²⁺-imaging of living cells was carried out using CaTM-3-AM. In order to study the utility thereof, a comparison was made with CaTM-2-AM(N-[2-[(acetyloxy)]-2-oxoethyl]amino]-N-[2-[2-[2-[bis[2-[(acetyloxy)methoxy]-2-oxoethyl]amino]-5-[[4-(1,8-dichloro-2,9-dihydro-7-hydroxy-9,9-dimethyl-2-oxo-9-silaanthracen-10-yl)-3-methylbenzoyl]amino]phenoxy]ethoxy]phenyl]-glycine(acetyloxy)methyl ester), which has a methyl group at position 2 of the benzene ring instead of a carboxy group. HeLa cells were incubated for 30 minutes at 37° C. together with 3 μM of CaTM-2-AM or CaTM-3-AM in a Hank's balanced salt solution (HBSS) containing 0.03% DMSO. The cells were washed three times using HBSS, then HBSS was added and the cells were observed using a confocal microscope SP5 (Leica) with an excitation wavelength of 590 nm and a fluorescence wavelength of 610 to 680 nm. The results are shown in FIG. 5.

It is apparent from FIG. 5 that CaTM-3-AM in which a carboxy group is introduced at position 2 of the benzene ring produces a brighter fluorescence image than does CaTM-2-AM. This shows that a larger quantity of fluorescent probes is taken into cells using CaTM-3-AM.

Ca²⁺ imaging was carried out during stimulation by histamine and ionomycin using cells to which CaTM-3-AM had been added. Histamine hydrochloride (1 μM) was added after 1 minute from the beginning of observation, and ionomycin (5 μM) was added after 4 minutes from the beginning of observation. The results are shown in FIG. 6. FIG. 7 shows the fluorescence images at each time point a, b, and c in FIG. 6. FIGS. 6 and 7 show that variation in Ca²⁺ in the cell during stimulation by histamine and ionomycin can be accurately ascertained by using CaTM-3-AM.

1. A compound represented by formula (I) below, or a salt thereof 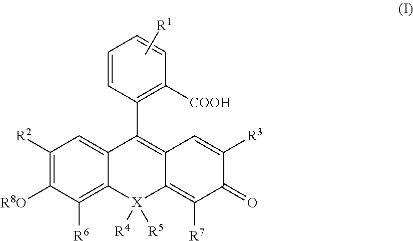
where:
R¹ represents 1 to 4 monovalent substituents present on a benzene ring, which are the same or different, where at least one of the substituents acts as a capturing group in relation to a substance to be measured;
R² and R³ are, independently, a hydrogen atom, a C₁₋₆ alkyl group, or a halogen atom;
R⁴ and R⁵ are, independently, a C₁₋₆ alkyl group or an aryl group;
R⁶ and R⁷ are, independently, a hydrogen atom, a C₁₋₆ alkyl group, or a halogen atom;
R⁸ is a substituent which acts as a capturing group in relation to the substance to be measured, a hydrogen atom, an alkylcarbonyl group, or an alkylcarbonyloxymethyl group; and
X is a silicon, germanium, or tin atom.

2. The compound or salt thereof according to claim 1, wherein X is a silicon or germanium atom.

3. The compound or salt thereof according to claim 1, wherein the capturing group of R¹ is a capturing group for capturing a proton, a metal ion, a low-oxygen environment, or a reactive oxygen species.

4. The compound or salt thereof according to claim 1, wherein the capturing group of R¹ is a capturing group for capturing a calcium ion.

5. The compound or salt thereof according to claim 1, wherein the capturing group of R¹ binds to a benzene ring via a spacer.

6. The compound or salt thereof according to claim 1 represented by formula (Ia) below: 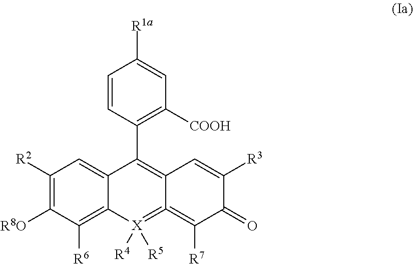
where:
R^{1a} is 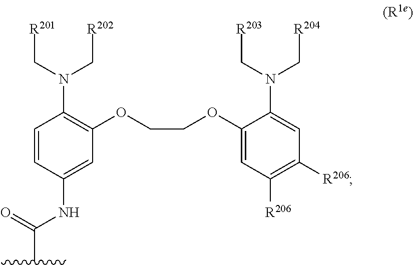
R² and R³ are, independently, a hydrogen atom, a C₁₋₆ alkyl group, or a halogen atom;
R⁴ and R⁵ are, independently, a C₁₋₆ alkyl group or an aryl group;
R⁶ and R⁷ are, independently, a hydrogen atom, a C₁₋₆ alkyl group, or a halogen atom;
R⁸ is a substituent which acts as a capturing group in relation to the substance to be measured, a hydrogen atom, an alkylcarbonyl group, or an alkylcarbonyloxymethyl group;
X is a silicon, germanium, or tin atom;
R²⁰¹, R²⁰², R²⁰³, and R²⁰⁴ are a carboxy group or an alkanoyloxyalkyloxycarbonyl group; and
R²⁰⁵ and R²⁰⁶ are, independently, a hydrogen atom, a C₁₋₆ alkyl group, a nitro group, or a halogen atom.

7. The compound or salt thereof according to claim 6 represented by formula (Ib) below: 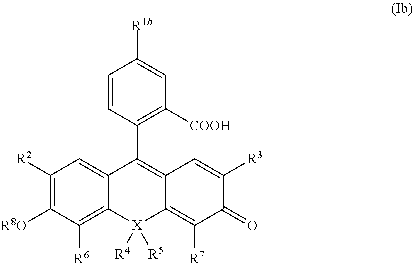
where:
R^{1b} is 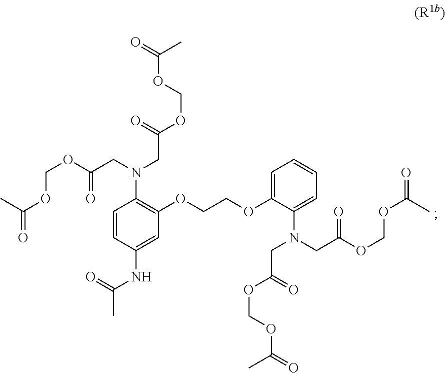 [SMILES: CC(=O)Nc1ccc(N(CC(=O)OCOC(C)=O)CC(=O)OCOC(C)=O)c(OCCOc2ccccc2N(CC(=O)OCOC(C)=O)CC(=O)OCOC(C)=O)c1]
R² and R³ are, independently, a hydrogen atom, a C₁₋₆ alkyl group, or a halogen atom;
R⁴ and R⁵ are, independently, a C₁₋₆ alkyl group or an aryl group;
R⁶ and R⁷ are, independently, a hydrogen atom, a C₁₋₆ alkyl group, or a halogen atom;
R⁸ is a substituent which acts as a capturing group in relation to the substance to be measured, a hydrogen atom, an alkylcarbonyl group, or an alkylcarbonyloxymethyl group; and
X is a silicon, germanium, or tin atom.

8. The compound or salt thereof according to claim 1, wherein the capturing group of R⁸ is a capturing group for capturing a proton, a reactive oxygen species, or a glycohydrolase.

9. A fluorescent probe comprising the compound or salt thereof according to claim 1.

10. A method for preparing a compound of formula (I): 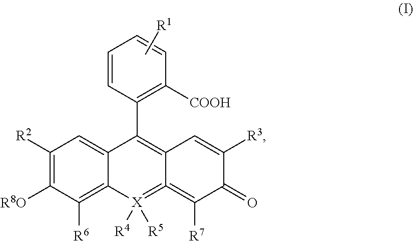
the method comprising:
(a) reacting 4-bromoisophthalic acid and t-butyl alcohol to obtain di-tert-butyl 4-bromoisophthalate; and
(b) reacting di-tert-butyl 4-bromoisophthalate and sec-butyl lithium, adding the compound represented by formula (II) below immediately thereafter 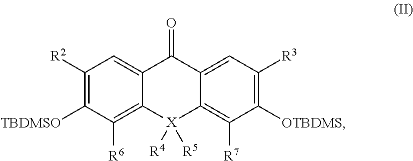
and then adding an acid to obtain a compound of formula (III) 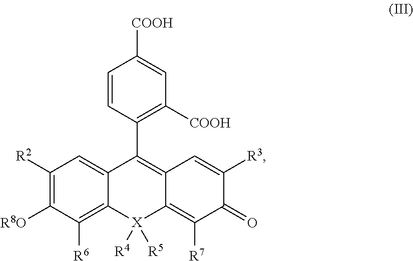
where in Formulas I-III,
R² and R³ are, independently, a hydrogen atom, a C₁₋₆ alkyl group, or a halogen atom;
R⁴ and R⁵ are, independently, a C₁₋₆ alkyl group or an aryl group;
R⁶ and R⁷ are, independently, a hydrogen atom, a C₁₋₆ alkyl group, or a halogen atom;
R⁸ is a substituent which acts as a capturing group in relation to the substance to be measured, a hydrogen atom, an alkylcarbonyl group, or an alkylcarbonyloxymethyl group; and
X is a silicon, germanium, or tin atom.

11. A method for measuring a substance to be measured, comprising:
(a) bringing the substance to be measured into contact with the compound or salt thereof according to claim 1; and
(b) measuring fluorescence intensity of the compound after capture of the substance to be measured generated in (a).
